(19) United States
(12) Patent Application Publication
(10) Pub. No.: US 2026/0284978 A1
(43) Pub. Date: Sep. 24, 2026

(54) Calibrating a Dispensing Means of a 3D Bio-Printer

(71) Applicant: Cellink Bioprinting AB, Göteborg (SE)

(72) Inventor: Rahul Suresh, Göteborg (SE)

(73) Assignee: Cellink Bioprinting AB, Göteborg (SE)

(21) Appl. No.: 19/476,211

(22) PCT Filed: Apr. 11, 2024
(86) PCT No.: PCT/SE2024/050344

§ 371 Date: Oct. 17, 2025

(30) Foreign Application Priority Data
Apr. 19, 2023 (SE) 2350469-9

(51) Int. Cl. B29C 64/393; B29C 64/209; B33Y 30/00; B33Y 50/02; G06T 7/73

(52) U.S. Cl. CPC B29C 64/393; B29C 64/209; G06T 7/73; B33Y 30/00; B33Y 50/02

(57) ABSTRACT

The present disclosure relates to a method, system, a 3D bio¬printer comprising control system, a computer program carrier and a computer program product. The computer-implemented method comprises obtaining sensor data representative of a printhead (1) of the 3D bio-printer, and a surrounding environment of the printhead, the obtained sensor data comprising one or more images of the printhead of the 3D bio-printer captured by means of an imaging sensor device (6). Further, the method comprises providing the obtained sensor data to a trained neural network algorithm. The method further comprises generating, by means of the neural network algorithm, one or more prediction outputs representative of a calibration condition of the dispensing means.

### TECHNICAL FIELD

The present disclosure relates to automated calibration of a dispensing nozzle of a 3D bio-printer. More specifically, the aspects and embodiments of the present disclosure relate to systems and methods for automatically calibrating the dispensing nozzle or needle of a 3D bio-printer by means of neural network models.

### BACKGROUND

During the last few years, the research and development activities related to 3-dimentional (3D) bio-printing have expanded in number and many different approaches are currently being explored. Amongst many technical and design parameter considerations and implementations of a 3D printing process is the ability to ensure a smooth and seamless flow of the printing process. To this end, an important step is to adequately calibrate the dispensing means of the 3D bio-printer which is configured to deliver the printing fluid to a printing bed and perform the printing of desired structures.

Conventionally, 3D bio-printers require a user to perform the calibration of the dispensing means e.g. a dispensing nozzle or a dispensing needle of the printer manually. However, there are several problems associated with the existing solutions. Experience shows that many users skip at least one step in the calibration process. For instance, it is a common and recurring issue to skip a dispenser positioning or priming step of the dispenser by the user, which may lead to issues with the extrusion of the printing fluid in the beginning of the printing or dispensing process and negatively affect the print quality. Furthermore, the dispenser may easily get contaminated during the manual calibration process, which should be meticulously avoided. In addition, the user has limited control over the whole calibration process making the calibration of the dispenser inconvenient and time-consuming for the user.

Accordingly, for elevated comfort, safety and accuracy of a printing process by a 3D bio-printer, there is a need for solutions in the art that are capable of calibrating the dispensing means of the 3D bio-printer in an automatically robust and reliable manner.

### SUMMARY

It is therefore an object of the present disclosure to provide a control system, a 3D bio-printer comprising such a control system, a method, a computer program carrier, and a computer program product which alleviate all or at least some of the drawbacks of presently known solutions.

More specifically, it is an object of the present disclosure to alleviate problems related to automated calibration of a dispensing means of the 3D bio-printer. The automated calibration throughout this description may also be referred to as one-click calibration indicating the fully automatized nature of the presented technology herein without any involvement from the user of the 3D bio-printer.

These objects are achieved by means of a control system, a 3D bio-printer comprising such a control system, a method, a computer program carrier, and a computer program product, as defined in the appended independent claims. The term exemplary is in the present context to be understood as serving as an instance, example or illustration.

According to a first aspect of the present disclosure there is provided a computer-implemented method for calibrating a dispensing means of a 3D bio-printer. The method comprises obtaining sensor data representative of a printhead of the 3D bio-printer, and a surrounding environment of the printhead, the obtained sensor data comprising one or more images of the printhead of the 3D bio-printer captured by means of an imaging sensor device. The sensor data comprises one or more sensor data points corresponding to the captured one or more images. The method further comprises providing the obtained sensor data to a trained neural network algorithm, wherein the method further comprises determining, by means of the neural network algorithm, a presence of the dispensing means on the printhead based on the obtained sensor data. In an instance that the presence of the dispensing means is determined, even further, the method comprises determining, by means of the neural network algorithm, an association of the dispensing means to one or more respective target object classes based on one or more characterizing features of the dispensing means. The method further comprises determining, at least partly by means of the neural network algorithm, an operational state of the dispensing means associated with the respective one or more target object classes based on a comparison with a quality-of-state classification scheme. Moreover, the method comprises determining, by means of the neural network algorithm, corresponding spatial coordinates of a dispensing outlet of the dispensing means with respect to a print bed of the 3D bio-printer. Even further the method comprises, generating, by means of the neural network algorithm, one or more prediction outputs representative of a calibration condition of the dispensing means.

In several embodiments, the dispensing means may comprise any one of a dispensing needle type or a dispensing nozzle type.

In various embodiments and aspects, the trained neural network algorithm may comprise a convolutional neural network and/or a transformer neural network algorithm configured for determining one or more calibration conditions of the dispensing means.

Accordingly, the present inventors have realized that by using utilizing deep learning neural networks trained on supervised image data to detect, identify, prime and calibrate the dispenser before starting the print process, a significantly flexible and efficient automated calibration of the dispenser of the bio printer can be achieved. The proposed systems and methods provide greater accuracy in image classification, sematic segmentation, object detection, simultaneous localization and mapping compared to traditional computer vision techniques. Using the systems and method proposed herein requires less expert analysis and fine tuning since the one-click calibration takes away the user involvement and human error factors. Even further, the proposed systems, methods and algorithms e.g. training algorithms for the neural networks can be readily fine-tuned by collected supervised data and better control over data parameters and output can thus be achieved. Due to reduced involvement of the code and debugging issues contrary to the conventional solutions, the solutions proposed herein make the process resistance to algorithmic logic bias. Accordingly, some the advantages achieved by the proposed solutions herein include:
The entire calibration process is a one click process, doesn't need any user input, and is ready to go from dispenser attachment to printing
No user input information is required
Much faster than conventional approaches
Uses a single sensor approach and supervised labelled data optimized for specified needle/nozzle dispenser calibration

In some embodiments, each target object class may be associated with one or more annotated data sets representative of that target object class, and wherein the one or more annotated data sets may be representative of at least one annotated characterizing feature of the dispensing means. The one or more characterizing features may comprise any one of a type of dispensing means, a colour indicator of the dispensing means, a size or a length measure of the dispensing means, or a transparency measure of the dispensing means.

In some embodiments, the quality-of-state classification scheme may comprise one or more quality-of-state classes, each class being associated with one or more annotated data sets representative of a respective quality of the dispensing means. The method may further comprise determining the operational state of the dispensing means by determining an association of the obtained sensor data with at least one quality-of-state class. In some embodiments, the one or more obtained images may comprise an ambient lighting and/or a shadow profile of the printhead and/or of the dispensing means when being present on the printhead and/or of the surrounding environment of the printhead.

In some embodiments and aspects, the ambient lighting and/or the shadow profile of the surrounding environment of the printhead may comprise an ambient lighting and/or a shadow profile of one or more reference markers arranged in the surrounding environment of the printhead.

In several embodiments herein, the method may further comprise determining the corresponding spatial coordinates of the dispensing outlet of the dispensing means based at least partly on the ambient lighting and/or the shadow profile of the printhead and/or of the dispensing means when being present on the printhead and/or of the surrounding environment of the printhead comprising the ambient lighting and/or a shadow profile of the one or more reference markers.

In some embodiments, the method may further comprise providing the obtained sensor data to an image analysis algorithm configured to determine an orientation of the dispensing means based on the obtained sensor data. In several embodiments, the method may further comprise determining, by means of the neural network algorithm, respective spatial coordinates of each sensor data point comprised in the sensor data. The method may further comprise determining the corresponding spatial coordinates of the dispensing outlet of the dispensing means based on the determined respective spatial coordinates for at least one sensor data point.

According to some exemplary embodiments, the method may further comprise determining, by means of the neural network algorithm, when a printing fluid in connection with the printhead is extruded from the outlet of the dispensing means. Further, when it is determined that the printing fluid is extruded from the outlet of the dispensing means, the method may further comprise terminating the extrusion of the printing fluid and generating a prediction output representative of the calibration condition indicative of an achieved priming of the outlet of the dispensing means.

According to a second aspect of the present disclosure there is provided a computer-readable storage medium comprising instructions which, when executed by one or more processors of a computing device of a 3D bio-printer, causes the computing device to carry out the method according to any one of the embodiments of the method of the first aspect.

According to a third aspect of the present disclosure there is provided a computer program product comprising instructions which, when the program is executed by one or more processors of a computing device of a 3D bio-printer, causes the computing device to carry out the method according to any one of the embodiments of the method of the first aspect.

According to a fourth aspect of the present disclosure there is provided a system for calibrating a dispensing means of a 3D bio-printer. The system comprises processing circuitry configured for obtaining sensor data representative of a printhead of the 3D bio-printer, and a surrounding environment of the printhead. The obtained sensor data comprises one or more images of the printhead of the 3D bio-printer captured by means of an imaging sensor device. The sensor data comprises one or more sensor data points corresponding to the captured one or more images. The processing circuitry is further configured for providing the obtained sensor data to a trained neural network algorithm. The processing circuitry is further configured for determining, by means of the neural network algorithm, a presence of the dispensing means on the printhead based on the obtained sensor data. In an instance that the presence of the dispensing means is determined, the processing circuitry is further configured for determining, by means of the neural network algorithm, an association of the dispensing means to one or more respective target object classes based on one or more characterizing features of the dispensing means. Further, the processing circuitry is configured for determining, at least partly by means of the neural network algorithm, an operational state of the dispensing means associated with the respective one or more target object classes based on a comparison with a quality-of-state classification scheme. Even further, the processing circuitry is configured for determining, by means of the neural network algorithm, corresponding spatial coordinates of a dispensing outlet of the dispensing means with respect to a print bed of the 3D bio-printer. Further, the processing circuitry is configured for generating, by means of the neural network algorithm, one or more prediction outputs representative of a calibration condition of the dispensing means.

According to a fifth aspect of the present disclosure there is provided a 3D bio-printer comprising one or more printheads, each printhead comprising a dispensing means having an outlet for extruding a printing fluid; and a system according to various embodiments of the fourth aspect herein for calibrating the dispensing means of a 3D bio-printer.

Further embodiments of the different aspects are defined in the dependent claims.

It is to be noted that all the embodiments, elements, features and advantages associated with the first aspect also analogously apply to the second, third, fourth and fifth aspects of the present disclosure.

These and other features and advantages of the present disclosure will in the following be further clarified in the following detailed description.

### BRIEF DESCRIPTION OF THE DRAWINGS

Further objects, features and advantages of embodiments of the disclosure will appear from the following detailed description, reference being made to the accompanying drawings. The drawings are not to scale.

FIG. 1 is a schematic perspective view illustration of a printhead and a control system in accordance with several embodiments of the present disclosure.

FIG. 2a is a schematic block diagram illustrating embodiments of a system of the present disclosure.

FIGS. 2b-2c are schematic perspective view illustrations of an image acquisition set up in accordance with several other embodiments of the present disclosure.

FIG. 3 is a schematic flowchart illustrating a method in accordance with several embodiments of the present disclosure.

FIG. 4 is a schematic side view illustration of a 3D bio-printer comprising a control system in accordance with several embodiments of the present disclosure.

### DETAILED DESCRIPTION

Those skilled in the art will appreciate that the steps, services and functions explained herein may be implemented using individual hardware circuitry, using software functioning in conjunction with a programmed microprocessor or general purpose computer, using one or more Application Specific Integrated Circuits (ASICs) and/or using one or more Digital Signal Processors (DSPs). It will also be appreciated that when the present disclosure is described in terms of a method, it may also be embodied in one or more processors and one or more memories coupled to the one or more processors, wherein the one or more memories store one or more programs that perform the steps, services and functions disclosed herein when executed by the one or more processors.

In the following description of exemplary embodiments, the same reference numerals denote the same or similar components.

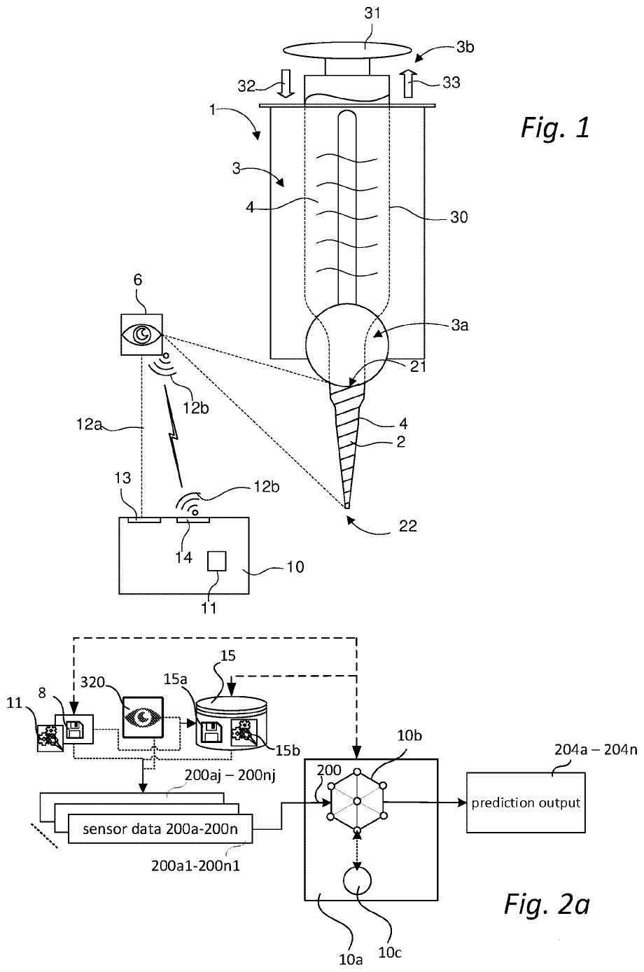

FIG. 1 illustrates a schematic perspective view of a printhead 1 of a 3D bio-printer 100 and a dispensing means 2 of the 3D bio-printer 100 which is comprised in the printhead 1. The dispensing means in the present context comprises any one of a dispensing means of a needle type or a dispensing means of a nozzle type, which will collectively be referenced as the dispensing means 2 or the dispenser 2 in the rest of this description.

The printhead 1 could be any suitable type of printhead for a 3D bio-printer. In this example, the printhead 1 comprises a plunger unit 3 housed within the printhead 1 and adapted to operate as means for fluid-administration 3. The plunger unit 3 at its distal end 3a is arranged to be connected to the dispensing means 2. The plunger unit 3 at its proximal end 3b is provided with a driver element 31, which may generally be referred to as a piston 31. The plunger unit 3 in its simplest form may be a syringe comprising a container 30 containing a printing fluid 4 and a piston 31 configured to apply a positive pressure 32 i.e. a drive, inject or introduce force 32 on the printing fluid 4 being introduced into the dispensing nozzle 2 by the introduce force 32 from the piston 31. The piston 31 of the plunger unit 3 may also be configured to apply a negative pressure 33 i.e. a pull, or retract force 33 on the printing fluid 4 inside the container 30 plunger unit 3 in order to stop an injection of the printing fluid 4 into the dispensing means 2 as well as retracting the printing fluid 4 further up into the dispensing means 2 or into the container 30 of the plunger unit 3 or into a separate cartridge unit (not shown) coupled to the plunger unit 3. In the context of the present disclosure when printing fluid 4 or material 4 is used it is to be construed as the substances and materials which are to be 3D-printed by the 3D bio-printer and are introduced into the cartridge or plunger unit e.g. by a user of the apparatus. To this end, the printing fluid 4 may comprise external objects, including but not limited to any one of at least one living cell, at least one bead such as spherical plastic beads, fluorescent beads used for tracing, etc., at least one organoid i.e. aggregates of living cells, or at least one spheroid i.e. spherically-shaped aggregates of living cells. The printing fluid 4 may comprise any one of a bioink, a printing ink, a hydrogel, a chemical reagent, a cell medium, crosslinking agents, and a gel. Examples of bioink may comprise any one of collagen, ColMA, GelMA, GelXA, alginate, pluronic, nanofibrillated cellulose, HAMA, Cellink, etc.

In this description, when using printing fluid it should be appreciated that it encompasses any one, or any combination of the above examples of the printing fluid 4 as well as embodiments where external objects may be present in the printing fluid to be printed.

The plunger unit 3 may in several embodiments be coupled at one end to a separate cartridge of the 3D printer containing the printing fluid 4. In some embodiments, the plunger unit 3 may be employed as the sole cartridge of the 3D bio-printer 100. For convenience, in the rest of this description, the plunger unit 3 and printing cartridge 3 may be used interchangeably. It is clear to the skilled person that various types and configurations of printheads and cartridges may be designed and installed into the 3D printers, the details of which will be omitted herein as to not obscure the advantageous aspects and embodiments of the presented system and method.

The plunger unit 3 is at its distal end 3a coupled to an inlet 21 of the dispensing means 2 enabling the injection of the printing fluid 4 comprised in the container 30 of the plunger unit 3 into the dispensing means 2. By the drive force 32 of the piston 31, the printing fluid 4 is injected introduced into the dispensing means 2 from the inlet 21 and is caused to flow towards and to be extruded from an outlet 22 of the dispensing means 2. The dispensing means 2 may in various embodiments be a needle suitable to be screwed onto the distal end 3a of the plunger unit 3 or a pipette tip 2 or any other suitable means for extruding the printing fluid from the container 30. The outlet 22 of the dispensing means 2 may also be referred to as an orifice 22 or opening 22 of the dispensing means 2 in the rest of this description.

FIG. 1 illustrates a system 10 which may also be referred to as a control system 10 or a control device 10 comprising processing circuitry 11 configured to perform automated calibration of the dispensing means 2 of the 3D bio-printer in accordance with several aspects and embodiments of the present disclosure.

More specifically, the processing circuitry 11 is configured for obtaining sensor data representative of the printhead 1 of the 3D bio-printer 100, and/or a surrounding environment of the printhead 1. The obtained sensor data (assuming “a-n” image acquisitions, “a” and “n” being real numbers denoting the instances of image acquisition and the associated sensor data with each image acquisition) 200a-200n comprises sensor data associated with one or more images of the printhead 1 including one or more images of the dispenser 2, and/or one or more images of the surrounding environment of the 3D bio-printer 100 including the surrounding environment of the printhead 1, captured by means of an imaging sensor device 6.

The one or more obtained images may comprise an ambient lighting and/or a shadow profile of the printhead 1 and/or of the dispensing means 2 when being present on the printhead 1 and/or of the surrounding environment of the printhead 1. The ambient lighting and/or the shadow profile of the surrounding environment of the printhead 1 may comprise an ambient lighting and/or a shadow profile of one or more reference markers 303a-303e arranged in the surrounding environment of the printhead 1.

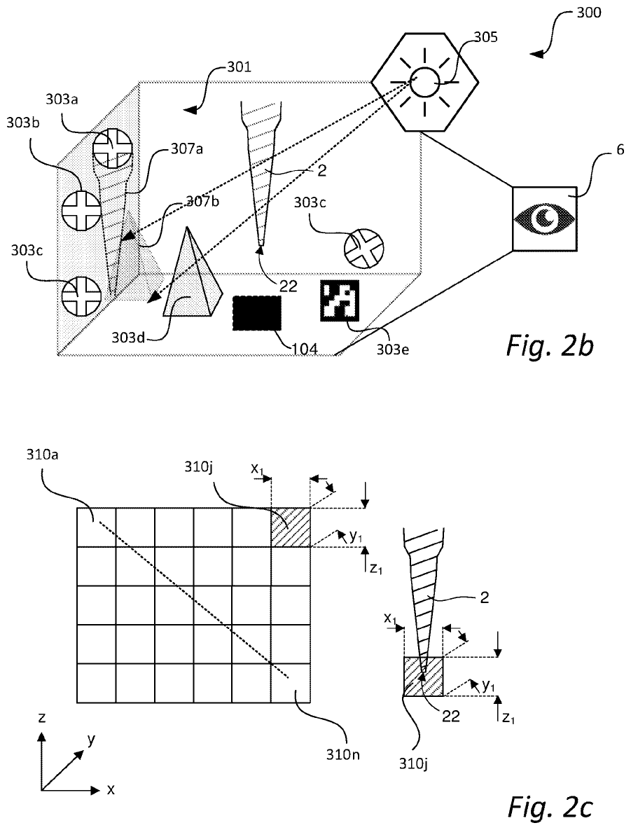

As shown in the example embodiment of FIG. 2b, a setup 300 for sensor data acquisition for training of the NN algorithm 10b and calibration of the dispensing means 2 may comprise a semi-closed background 301 with one or more exemplary reference markers 303a-303e in the background 301. In several embodiments, the reference markers 303a-303e may be placed between the dispenser 2 and the imaging sensor device 6.

In some embodiments, a 3D marker 303d such as a pointer or any other figure may be arranged in the surrounding environment of the printhead 1 and used as the reference marker 303d. Additionally or alternatively, a fiducial marker (object placed in the field of view of an imaging system that appears in the image produced, for use as a point of reference or a measure. It may be either something placed into or on the imaging subject, or a mark or set of marks in the reticle of an optical instrument) could also be used as a reference marker. Even more additionally or alternatively, QR Codes/ArUco markers such as exemplary marker 303e may be used as reference marker, for instance QR marker 303e placed between the dispenser 2 and the imagining sensor device 6 in FIG. 2b. It should be appreciated that the illustrated reference markers such as the cross-shaped reference markers are simply for illustrative purposes and reference markers of other shapes and orientations may be placed in the surrounding environment of the printhead 1. Analogously, various other 3D markers or pointers than the depicted marker 303d in FIG. 2b may be employed.

A singular or multiple light setups 305 (only one illumination source 305 shown in FIG. 2b) may be used to create ambient lighting and shadows 307a, 307b of the needle/nozzle dispenser 2 or the one or more reference markers 303a-303e which will be employed in the calibration setup 300. In some embodiments, the imaging sensor device 6 may be calibrated by utilizing the reference markers 303a 303e.

The sensor data based on a-n image acquisitions 200a-200n comprises one or more sensor data points 200a1-200aj, 200b1-200bj, . . . , 200n1-200nj corresponding to respective captured one or more images. For example, image “a” may correspond to respective sensor data 200a which may comprise one or more sensor data points 200a1-200aj, wherein “j” is a real number simply used for indicating one or more sensor data points being comprised in each respective sensor data 200a-200n. Each image may be represented by a plurality of pixels. In several embodiments, each pixel may be represented by and correspond to one respective sensor data point comprised in the obtained sensor data 200a-200n. In some embodiments, each pixel may be represented by and correspond to more than one respective sensor data point i.e. a plurality of sensor data points comprised in the obtained sensor data 200a 200n.

The obtained one or more images also comprises sensor data points representing the dispensing means 2. The imaging sensor device 6 may be a stand-alone unit configured to be connectable to the control system 10 and in communication with the processing circuitry 11 via wired 12a and/or wireless 12b communication links. Alternatively or additionally, the imaging sensor device 6 may be comprised in the control device 10 and/or in the 3D bio-printer e.g. as a part or module of the 3D bio-printer. This is e.g. shown in FIG. 5, wherein the imaging sensor device 6 is a component of the control device 10 and/or the 3D bio-printer 100. The imaging sensor device 6 can be connected to the control system 10 and in communication with the processing circuitry 11 via wired 12a and/or wireless 12b communication. In several embodiments, the imaging sensor device 6 is one of a fixed-focused camera 6 or an auto-focus camera 6 capable of operating in the visible or infrared spectrum. The imaging sensor device 6 may obtain images of the whole body of the dispensing means 2 including images of the outlet 22 of the dispensing means 2 as well as images of the printing fluid 4 inside the dispensing means 2 (e.g. when the dispensing means 2 is made of transparent materials in the visible spectrum of the camera) and extruded from the orifice 22 of the dispensing means 2. The imaging sensor device 6 may be connected to the control system 10 via a designated sensor interface 13 comprised in the control system 10. In various aspects and embodiments, the control system 10 may be a standalone entity in communication with the 3D bio-printer 100 and arranged remotely from the 3D bio-printer 100 e.g. at a remote control centre or be implemented on a cloud communication network 20. In various aspects and embodiments such as the embodiment shown in FIG. 5, the control system 10 may be comprised in the 3D bio-printer 100 as a component or module of the 3D bio-printer 100.

As shown in the block diagram of FIG. 2a, the obtained sensor data 200a-200n associated with the one or more images captured by the imaging sensor device 6 is provided to a trained neural network (NN) algorithm 10b as input data 200. In several embodiments, the trained neural network algorithm 10b may comprise a convolutional neural network (CNN) algorithm configured for determining one or more calibration conditions of the dispensing means 2. Additionally or alternatively, the trained neural network algorithm 10b may comprise a transformer neural network used for both classification and object detection. A transformer model in the present context is a neural network that learns context and thus meaning by tracking relationships in sequential data.

In several aspects and embodiments, the imaging sensor device 6 may be in communication with the trained NN algorithm 10b. The trained NN algorithm 10b may be comprised in a component of the control system 10 such as a monitoring unit 10a adapted to monitor the surrounding environment of the printhead 1 and the dispensing means 2 based on the obtained sensor data 200a-200n. In some embodiments, the NN algorithm 10b may at least partly be comprised in or be implemented by the processing circuitry 11. The NN algorithm 10b mat be stored in a memory 8 of the control system 10. The monitoring unit 10a in some embodiments may be collocated with or be comprised in the imaging sensor unit (not shown). The monitoring unit 10a in some embodiments may be comprised in the processing circuitry 10a e.g. as a component or module of the processing circuitry. The imaging sensor device 6 may transmit, S1, the obtained one or more images of the dispensing means 2 to the monitoring unit 10a for providing the sensor data as input data 200 to the NN algorithm 10b. As mentioned above, the monitoring unit 10a and/or the control system 10 in several embodiments may comprise at least one memory or storage mediums 8 to store the NN algorithm 10b. In several embodiments, the monitoring unit 10a might be partly or completely realized in software and be located on a cloud network 20 and accessed by the control system 10 wirelessly.

The neural network algorithm 10b may have been previously trained with a training data set and stored in the memory 8 or in a data storage 15a of a remote server 15 for subsequent execution. In several embodiments, the neural network algorithm 10b may be continuously and repeatedly trained with updated training data. The training data may be obtained and curated based on various data collection and classification approaches. The training data may be obtained from the server 15, and may be formed based on the information of the surrounding environment of the printhead 1. In several embodiments and aspects, the training data may at least partly be formed based on sensor data obtained by the sensor device 6.

In some examples, the neural network algorithm 10b has been trained on training data at least partly formed based on sensor data obtained by the imaging sensor device.

Various aspects and embodiments of the presented technology, and the corresponding systems and methods may be implemented and executed by means of processing circuitry comprising one or more processors. The processing circuitry may at least partly be comprised in a control system 15b or apparatus 15b implemented in the data centre 15 or server 15. In some embodiments and aspects, the control system 10 or apparatus 10 of the 3D bio-printer 100 may be configured to perform the action and functions described herein.

The processing circuitry 11 of the control system 10, 15b may thus be further configured for determining, by means of the neural network algorithm 10b, a presence of the dispensing means 2 on the printhead 1 based on the obtained sensor data 200a-200n. In other words, the NN algorithm 10b is trained to identify based on the input data 200 i.e. the one or more sensor data points 200a1 200aj, 200b1-200bj, . . . , 200n1-200nj, if the dispensing means 2 is in fact installed and is present on the printhead 1 at the start of the automated calibration process. In several embodiments, if the presence of the dispensing means cannot be determined, an error or warning signal may be generated based on the prediction output 204a-204n of the NN algorithm 10b indicating the absence of the dispensing means 2.

In several embodiments, only one detection and determination of presence of the dispenser 2 may be carried out, whereas in some embodiments multiple detections may be done. The output provided by the NN algorithm 10b may either be confirming a presence of the dispense 2 i.e. needle or nozzle or a prediction of dispenser 2 not present at the printhead 1 may be generated. Supervised data comprising different dispenser needles and/or nozzles 2 with specific prominent features such as colour, size, transparency, etc. may be collected and neural network-based object detection algorithm 10b applied. The NN algorithm 10b may have two classes i.e., background with no dispenser nozzle/needle 2 and another object class with the nozzle and/or needle 2 present. The outcome of the NN algorithm 10b may thus a binary sequence e.g. yes—(1), for the prediction output 204a-204n of needle/nozzle 2 being present or no—(0), for the prediction output 204a-204n of the needle/nozzle 2 being absent. In several embodiments, the detection may be performed in a frame-to-frame sequence applied to the image sensor data.

In some embodiments, as a consequence of the generated prediction output 204a-204n no—(0), the automated calibration of the dispensing means 2 may be interrupted until the user attached a dispensing means 2 to the printhead 1 and restarts the calibration process. In some embodiments, an error signal may be generated and displayed to the user on a display means of the bio-printer 102 or on a graphical user interface (GUI) through which the user interacts with the control system 10, 15b.

In an instance that the presence of the dispensing means 2 is determined, the processing circuitry 11 of the control system 10, 15b is further configured for determining, by means of the neural network algorithm 10b, an association of the dispensing means 2 to one or more respective target object classes based on one or more characterizing features of the dispensing means. Here the detected dispensing means 2 such as a dispensing needle 2 or dispensing nozzle 2 are classified based on their characteristics and it is determined to which object classes they belong. The object classes may in several embodiments comprise one or more characterizing features of the dispensing means 2 such as physical attributes of the nozzle or needle, size of the nozzle or needle, shape of the dispenser 2, various lighting or colour nuances etc.

In some examples, determining, by means of the neural network algorithm 10b, the association of the dispensing means 2 to one or more respective target object classes based on one or more characterizing features of the dispensing means, is further based on the obtained sensor data and/or data indicative of the characterizing features of the dispensing means stored in a memory 8 of the control system 10.

In several embodiments, each target object class may be associated with one or more annotated data sets representative of that target object class. The one or more annotated data sets are thus representative of at least one annotated characterizing feature of the dispensing means 2. The one or more characterizing features as mentioned above may comprise any one of a type of dispensing means 2, a colour indicator of the dispensing means 2, a size or a length measure of the dispensing means 2, or a transparency measure of the dispensing means 2.

Hence, after detection of the dispenser 2 is determined, a classification step of the detected needle and/or nozzle 2 may be performed by the NN algorithm 10b and the processing circuitry 11. As mentioned above, the classification may be based on attributes such as the colour of the dispenser 2. The colour attribute may comprise sub-classes such as transparent, red, blue, green, etc. In some embodiments, the classification may be based on a size or length attribute of the needle and/or nozzle 2 e.g. 0.25, or 0.5, or 0.75 inch, etc.

A similar supervised data set used in the detection step may be used and the object classification algorithm 10b may be used to classify the detected needle/nozzle 2 based on respective attributes. The number of classes related to the attributes and characteristics would be depending on the needle/nozzle 2 and may comprise a plurality of object classes. The prediction output 204a-204n of the NN algorithm 10b may accordingly be a specific needle and/or nozzle 2 with an associated colour, size, or length, etc. It should be appreciated that this classification process will be an instantaneous classification and will happen once the dispenser 2 is detected, and all without any user involvement.

The processing circuitry 11 of the control system 10, 15b is further configured for determining, at least partly by means of the neural network algorithm 10b, an operational state of the dispensing means 2 associated with the respective one or more target object classes based on a comparison with a quality-of-state classification scheme.

In some examples, the processing circuitry 11 of the control system 10, 15b is further configured for determining, at least partly by means of the neural network algorithm 10b, an operational state of the dispensing means 2 associated with the respective one or more target object classes from a predetermined set of target object classes, such as classes of needles and nozzles.

In several embodiments and aspects, the quality-of-state classification scheme may comprise one or more quality-of-state classes, each class being associated with one or more annotated data sets representative of a respective quality of the dispensing means. The processing circuitry 11 of the control system 10, 15b may further be configured for determining the operational state of the dispensing means 2 by determining an association of the obtained sensor data 200a-200n with at least one quality-of-state class.

It is to be understood that determining the operational state of the dispensing means typically relates to comparing obtained sensor data to a finite number of operational states defined by a predetermined quality-of-state classification scheme. The comparison and/or the predetermined quality-of-state classification scheme may be based on the determined association of the dispensing means 2 to one or more respective target object classes, such as using different classification schemes if the dispensing means 2 has been determined to be a nozzle or determined to be a needle.

In several embodiments, an image processing algorithm 10c may be configured for processing the obtained one or more images of the printhead 1 and/or the dispensing means 2. In some embodiments, the image processing algorithm 10c may be comprised in the monitoring unit 10a. The one or more images may sometimes be referred to as raw image data. The processing circuitry 11 may pre-process and extract information from the raw image data, providing the NN algorithm 10b and/or the image processing algorithm 10c with the processed image sensor data or as otherwise referred to the sensor data. The image processing algorithm 10c may be selected based on the specific applications and capabilities of the processing circuitry and can be any suitable image processing algorithm known in the art. In some embodiments, additionally or alternatively the image processing algorithm 10c may receive the raw image data as input and produce in several steps, processed image sensor data. For example, a visible-spectrum colour camera capable of colour image acquisition of the dispensing means 2 may be configured to continuously obtains one or more images of the dispensing means 2. The raw colour images may then be converted to grayscale images. A Gaussian blur may be applied to the grayscale image to at least partly remove the noise in the grayscale image. An edge detection principle such as Canny edge detection may be used to detect the outline of the dispenser 2 or the dispenser outlet 22 in the image. Contour detection may be applied to locate the outline of the nozzle outlet 22. The extreme points (top, bottom, right, left) of the nozzle contour may then be calculated.

In several embodiments, the obtained sensor data 200a-200n would be provided to the NN algorithm 10b and/or the image analysis algorithm 10c configured to determine an orientation of the dispensing means 2 based on the obtained sensor data 200a-200n. In several embodiments, the sensor data is preliminarily provided to the image processing algorithm 10c. The output of the image processing algorithm 10c would denote the orientation of the dispenser 2 and will subsequently be provided to the NN algorithm 10b for further succeeding determination and classification steps.

Quality control for the detected needle and/or nozzle 2 may be performed to ensured that the detected specific dispenser 2 passes the quality parameters required prior to starting a print job by the bio-printer. Accordingly, the NN algorithm 10b alone, the image processing algorithm 10c, or the NN algorithm 10b in combination with the image processing algorithm 10c may perform the quality check of the detected nozzle and/or needle 2. It may thus be determined based on the quality-of-state classification scheme and various quality-of-state classes e.g. if the dispenser 2 belongs to a specific quality-of-state class such as faulty dispenser e.g. damaged, broken, clogged, or has bio-ink spilled over it or a needle dispenser 2 may be bent or disoriented, or tilted etc. The processing circuitry may also determine presence of air bubbles in the dispenser 2 using the NN algorithm 10b or the classical image analysis algorithm 10c.

The quality check of the dispenser 2 in some embodiments may be performed in a two-step process including the classical image analysis algorithm 10c determining the orientation of the needle/nozzle 2 in step one and a fault detection step that employs the NN algorithm 10b trained with supervised data for fault detection. As mentioned earlier, a CNN-based classification may be used with object classes named after different faults and good quality needle/nozzle. After quality control, air bubble detection may be performed for the dispenser 2. In several embodiments, the step of air bubble detection may be performed on the transparent/translucent dispensers 2. In some embodiments, the dispensing means 2 may be made of the transparent material such as Polypropylene (PP), High-Density Polyethylene (HDPE), Polycarbonate (PC), Polystyrene (PS), glass or the like. The NN algorithm 10b for object classification may further be trained on supervised data with classes associated with presence or absence of air bubbles. The air bubbles might have nested classes based on the size of the air bubbles.

The processing circuitry 11 of the control system 10, 15b is further configured for determining, by means of the neural network algorithm 10b, corresponding spatial coordinates of a dispensing outlet 22 of the dispensing means 2 with respect to a print bed 103 of the 3D bio-printer 100. Further, the processing circuitry 11 of the control system 10, 15b is configured for generating, by means of the neural network algorithm 10b, one or more prediction outputs 204a-204n representative of a calibration condition of the dispensing means 2.

It is to be understood that “the one or more prediction outputs representative of a calibration condition of the dispensing means” relates to an output that, either directly predicts, or can be used to determine, whether the dispensing means 2 are ready for use, or is in a calibration condition that is incompatible with use. In some examples, the prediction outputs may be utilized by subsequent methods, or method steps, to make the dispensing means ready for use.

In some examples, determining, by means of the neural network algorithm 10b, corresponding spatial coordinates of the dispensing outlet 22 of the dispensing means 2, is based on the obtained sensor data.

In several aspects and embodiments, the calibration condition of the dispensing means 2 may comprise any one of a calibrated and ready-to-print, error in calibration-process interrupted, user intervention to mitigate dispenser 2 installation defects required, recalibration required, and the like.

In several embodiments, the processing circuitry 11 of the control system 10, 15b may be further configured for determining, by means of the NN algorithm 10b, the corresponding spatial coordinates of the dispensing outlet 22 of the dispensing means 2 based at least partly on the ambient lighting and/or the shadow profile of the printhead 1 and/or of the dispensing means 2 when being present on the printhead 1 and/or based on the ambient lighting and/or the shadow profile of the surrounding environment of the printhead 1 comprising the ambient lighting and/or a shadow profile of the one or more reference markers 303a-303e.

In several embodiments, the processing circuitry 11 of the control system 10, 15b may be further configured for determining, by means of the NN algorithm 10b, respective spatial coordinates of each sensor data point comprised in the sensor data and determining the corresponding spatial coordinates of the dispensing outlet 22 of the dispensing means 2 based on the determined respective spatial coordinates for at least one sensor data point. In other words, one or more pixels in the image data or in a frame of the image sensor data may have an assigned coordinate to it, based on which the spatial coordinates of the tip 22 may be predicted by the NN algorithm 10b.

Estimating coordinates such as x, y, and z coordinates of the dispenser 2 i.e. the nozzle/needle outlet or tip 22 may accordingly be performed with reference to the print bed 103. The NN algorithm 10b may be specifically trained with supervised data sets which comprise coordinates of the tip 22 with specific image data in reference to the background and/or lighting in the one or more image frames in the image sensor data. Different lighting, shadows or markers in a guesstimate system might be used for training data. Posnet, xyzNet, PnP-Net are amongst examples of neural networks 10b that can be used for training and implementation of this calibration system.

In some embodiments, the bio-printer 100 may undergo a step referred to as homing step, bringing the printhead 1 to a specific position before automated calibration step is commenced. Approximate coordinate boundaries with an error margin of e.g. around 1 cm in all directions i.e. x, y and z axis may be known and assigned to the homing position. After the homing step, the control system 10, 15b, may be configured to direct the printhead1 to the calibration centre 104, otherwise referred to as the calibration bay 104. An image acquisition may be performed using the camera 6. In some cases, the homing step could be skipped depending on the equipment and intended applications. The prediction output 204a-204n of the NN algorithm 10b would be the exact coordinates of the tip 22. The NN algorithm 10b would be trained with images specific to the coordinates and once the obtained image sensor data matches the images forming the training data, the exact coordinates of the dispenser outlet 22 can be estimated. The NN algorithm 10b may be trained within a small volume e.g. having x, y, z dimensions of 1 cm×1 cm×1 cm or 0.5 cm×0.5 cm×0.5 cm and the like.

In several aspects and embodiments as for instance shown in FIG. 2c, one or more pixels 310a-310n in the obtained image data may be mapped to their respective coordinate system. Thus, when the tip 22 is determined to be positioned in a particular pixel box associated with a set of spatial coordinates, the coordinates of the tip 22 can be determined accordingly. For instance, the pixel 310j in FIG. 2c is associated with corresponding spatial coordinates x1, y1 and z1 and thus, when it is determined based on the sensor data point(s) associated with the pixel 310j that the tip 22 of the dispenser 2 is located therein, the coordinates of the tip 22 is accordingly determined. This method of training advantageously enables the NN algorithm 10b for generating precise prediction outputs 204a-204n. More specifically, each pixel of the obtained images in a 2D plane may represent the x, and y coordinates. This image data may be used for training the NN algorithm 10b and would be used for generating straightforward and rapid prediction outputs 204a-204n by the NN algorithm 10b. The depth i.e., z coordinate may be estimated using supervised monocular depth estimating which may employ both the NN algorithm 10b and the image analysis algorithm 10c. By means of this advantageous combination of the NN algorithm 10b and the image processing algorithm 10c, x, y and z coordinates of the dispenser outlet 22 may be readily estimated and used in the calibration process. As mentioned above, the NN algorithm 10b may be trained on features and attributes of the image sensor data such as a shadow profile of the one or more reference markers, proximity of the one or more reference markers 330a-330d to the outlet 22 of the dispenser 2, background 301, etc.

In several embodiments, the processing circuitry 11 of the control system 10, 15b may be further configured for determining, by means of the neural network algorithm 10b, when the printing fluid 4 in connection with the printhead 1 is extruded from the outlet 22 of the dispensing means 2. When it is determined that the printing fluid 4 is extruded from the outlet of the dispensing means, the processing circuitry 11 is further configured for terminating the extrusion of the printing fluid 4 and generating a prediction output 204a-204n representative of the calibration condition indicative of an achieved priming of the outlet 22 of the dispensing means 2. In several embodiments and aspects, machine learning algorithms such as Random Forest may be used in the volume calculation of the printing fluid 4 such as bio ink at the tip 22 of the dispenser 2 during the priming step. After priming the dispenser tip 22, a volume of the printing fluid 4 at the nozzle/needle tip 2 may be estimated by means of ML algorithms. In some embodiments, the estimated amount of the printing fluid 4 at the nozzle/needle tip 2 may be retracted into the dispenser partly or entirely in order to minimize the wastage percentage of the printing fluid 4.

Accordingly, in some embodiments, when the processing circuitry and the NN algorithm 10b generate a prediction output 204a-204n indicating a calibrated status of the dispenser 2, wherein the dispenser 2 is ready to perform the print job, the priming step may be performed. In some embodiments, annotated image data of primed and non-primed dispenser outlet 22. The annotated image data may be used for training the NN algorithm 10b for detecting priming of the outlet 22 of the dispenser 2. Classical detection-classification problem that may thus be easily solved using the NN algorithm 10b for automated priming of the outlet 22 of the dispenser 2. In some embodiments, the processing circuitry may further be configured to commence a printing process by the 3D bio-printer after terminating the extrusion of the printing fluid out of the outlet of the dispensing means and determining that the priming of the outlet of the dispensing means has occurred.

It is to be understood that the term neural network algorithm relates to an algorithm with one or more neural networks. Thus, the neural network algorithm is not limited to containing one single neural network arranged to create an output containing “presence of the dispensing means”, “target object class” and “operational state of the dispensing means”, “spatial coordinates”, and “prediction outputs”. In some examples, the neural network algorithm comprises at least one neural network trained to output each of said “presence of the dispensing means”, “target object class” and “operational state of the dispensing means”, “spatial coordinates”, and “prediction outputs”. It is further to be understood that, determining or generating “by means of the neural network algorithm” does not condition that the output of a neural network in the algorithm is the same as the output of the algorithm itself, instead the output of a neural network may be processed by traditional techniques to determine or generate said “presence of the dispensing means”, “target object class” and “operational state of the dispensing means”, “spatial coordinates”, and/or “prediction outputs”.

In some examples, the neural network algorithm comprises two or more neural networks. In some of these examples, the output of one neural network in the algorithm serves as the input for one or more other neural networks in the algorithm. In some examples, the output of one neural network in the algorithm may be utilized to determine or generate at least two of said “presence of the dispensing means”, “target object class” and “operational state of the dispensing means”, “spatial coordinates”, and/or “prediction outputs”.

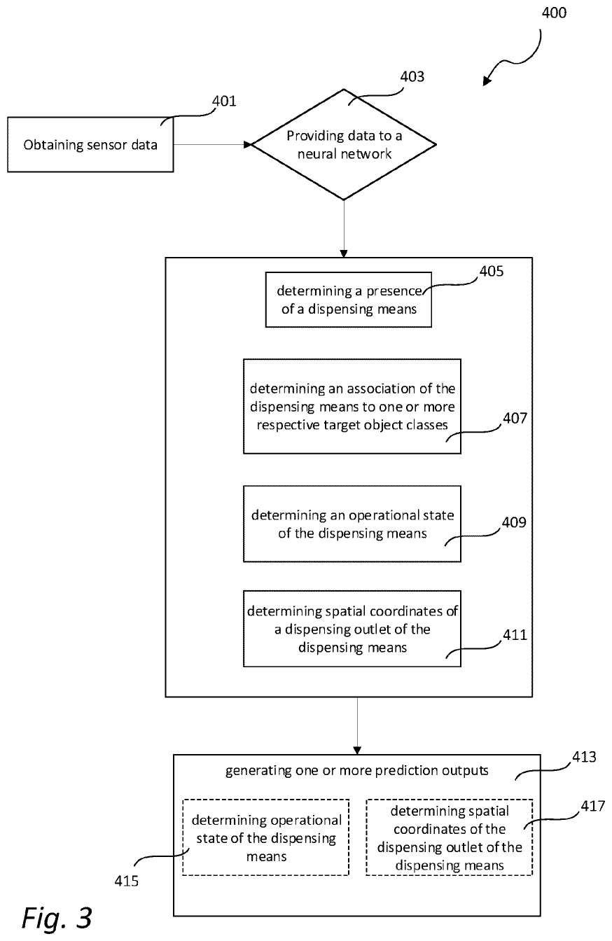

FIG. 3 shows a flow chart of a method 400 according to an aspect of the present disclosure for calibrating a dispensing means 2 of a 3D bio-printer 100. The computer-implemented method 400 comprises obtaining 401 sensor data representative of a printhead 1 of the 3D bio-printer 100, and a surrounding environment of the printhead 1. The obtained sensor data 200a-200n comprises one or more images of the printhead 1 of the 3D bio-printer 100 captured by means of an imaging sensor device 6. The sensor data comprises one or more sensor data points corresponding to the captured one or more images. The method 400 further comprises providing 403 the obtained sensor data 200a-200n to a trained neural network algorithm 10b. The method 400 further comprises determining 405, by means of the neural network algorithm 10b, a presence of the dispensing means 2 on the printhead 1 based on the obtained sensor data 200a-200n. In an instance that the presence of the dispensing means 2 is determined, the method 400 comprises determining 407, by means of the neural network algorithm 10b, an association of the dispensing means 2 to one or more respective target object classes based on one or more characterizing features of the dispensing means 2. Further, the method 400 comprises determining 409, at least partly by means of the neural network algorithm 10b, an operational state of the dispensing means 2 associated with the respective one or more target object classes based on a comparison with a quality-of-state classification scheme. The method 400 further comprises determining 411, by means of the neural network algorithm 10b, corresponding spatial coordinates of a dispensing outlet of the dispensing means with respect to a print bed 103 of the 3D bio-printer 100. The method 400 further comprises generating 413, by means of the neural network algorithm 10b, one or more prediction outputs 204a-204n representative of a calibration condition of the dispensing means 2.

In several embodiments, the dispensing means 2 may comprise any one of a dispensing needle type or a dispensing nozzle type.

In some embodiments, the trained neural network algorithm 10b may comprise a convolutional neural network algorithm 10b configured for determining one or more calibration conditions of the dispensing means 2. Additionally or alternatively, the trained neural network algorithm 10b may comprise a transformer neural network used for both classification and object detection. A transformer model in the present context is a neural network that learns context and thus meaning by tracking relationships in sequential data.

In several embodiments, each target object class may be associated with one or more annotated data sets representative of that target object class, wherein the one or more annotated data sets may be representative of at least one annotated characterizing feature of the dispensing means 2. The one or more characterizing features may comprise any one of a type of dispensing means, a colour indicator of the dispensing means, a size or a length measure of the dispensing means, or a transparency measure of the dispensing means.

In some examples, determining, by means of the neural network algorithm 10b, the association of the dispensing means to one or more respective target object classes based on one or more characterizing features of the dispensing means 2, is further based on the obtained sensor data and/or stored data indicative of the characterizing features of the dispensing means.

In several embodiments, the quality-of-state classification scheme may comprise one or more quality-of-state classes, each class being associated with one or more annotated data sets representative of a respective quality of the dispensing means. The method may further comprise determining 415 the operational state of the dispensing means by determining an association of the obtained sensor data 200a-200n with at least one quality-of-state class.

In several embodiments, the one or more obtained images may comprise an ambient lighting and/or a shadow profile of the printhead and/or of the dispensing means when being present on the printhead and/or of the surrounding environment of the printhead. The ambient lighting and/or the shadow profile of the surrounding environment of the printhead 1 may comprise an ambient lighting and/or a shadow profile of one or more reference markers arranged in the surrounding environment of the printhead 1.

In some examples, determining, by means of the neural network algorithm 10b, corresponding spatial coordinates of the dispensing outlet 22 of the dispensing means 2, is based on the obtained sensor data.

In several embodiments, the method may further comprise determining 417 the corresponding spatial coordinates of the dispensing outlet 22 of the dispensing means 2 based at least partly on the ambient lighting and/or the shadow profile of the printhead and/or of the dispensing means when being present on the printhead 1 and/or of the surrounding environment of the printhead 1 comprising the ambient lighting and/or a shadow profile of the one or more reference markers.

In some embodiments, the method may further comprise providing 419 the obtained sensor data 200a-200n to an image analysis algorithm configured to determine an orientation of the dispensing means 2 based on the obtained sensor data 200a-200n.

In some embodiments, the method may further comprise determining 421, by means of the neural network algorithm 10b, respective spatial coordinates of each sensor data point comprised in the sensor data. The method may further comprise determining 423 the corresponding spatial coordinates of the dispensing outlet 22 of the dispensing means based on the determined respective spatial coordinates for at least one sensor data point.

In several embodiments, the method may further comprise determining 425, by means of the neural network algorithm 10b, when a printing fluid 4 in connection with the printhead 1 is extruded from the outlet 22 of the dispensing means 2. When it is determined that the printing fluid 4 may be extruded from the outlet 22 of the dispensing means, the method may further comprise terminating 427 the extrusion of the printing fluid 4 and generating 429 a prediction output 204a-204n representative of the calibration condition indicative of an achieved priming of the outlet 22 of the dispensing means 2.

All the elements, features, relations of features and advantages explained in relation to the other aspects and as explained with reference to FIGS. 1-3 also apply analogously to this aspect of the present disclosure.

Executable instructions for performing the above functions and features of the embodiments of the methods are, optionally, included in a non-transitory computer-readable storage medium or other computer program product configured for execution by one or more processors of the processing circuitry.

More specifically, there is provided a computer program carrier carrying one or more computer programs configured to be executed by one or more processors of a of a computing device of a 3D bio-printer, the one or more programs comprising instructions for performing any one of the embodiments of the method 400 according to this disclosure. The computer program carrier may be one of an electronic signal, optical signal, radio signal or a computer-readable storage medium.

Even further, there is provided a computer program product comprising instructions which, when the program is executed by one or more processors of a processing circuitry such as of a computing device of a 3D bio-printer, causes the processing circuitry to carry out any one of the embodiments of the method 400 according to this disclosure.

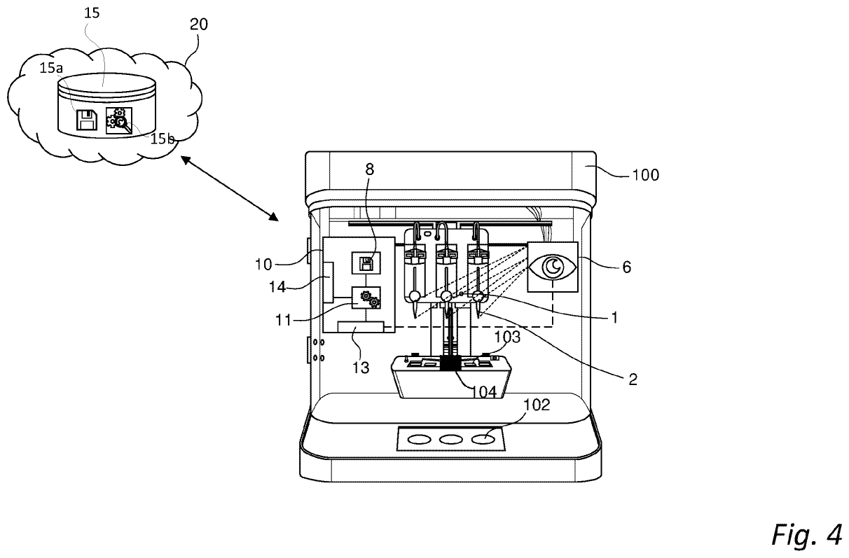

FIG. 4 is a schematic side view illustration of a 3D bio-printer 100 according to several aspects and embodiments of the present disclosure. The 3D bio-printer 100 comprises one or more print heads 1, wherein each printhead 1 comprising a dispensing means 2 having an inlet 21 for introduction of a printing fluid 4 into the dispensing means 2 and an outlet 22 for extruding the printing fluid 4 from the outlet 22 of the dispensing means 2. The 3D bio-printer 100 may comprise one or more imaging sensor devices 6 configured to obtain one or more images of the dispensing means 2 or of the printheads 1 and a surrounding environment of the printheads 1. In some embodiments, the 3D bio-printer 100 may be provided with the imaging sensor devices 6 as a separate entity being in communication with the 3D bio-printer 100. The 3D bio-printer also comprises a system 10 configured to perform any one of the embodiments of the methods 400 according to this disclosure.

The control system 10 comprises one or more processors 11, a memory 8, a sensor interface 13 and a communication interface 14. The processor(s) 11 may also be referred to as a control circuit 11 or control circuitry 11. The control circuitry 11 is configured to execute instructions stored in the memory 8 to perform several embodiments of the method 400 for calibrating the dispensing means 2 of the 3D bio-printer 100 according to any one of the embodiments disclosed herein. The memory 8 of the control system 10 can include one or more (non-transitory) computer-readable storage mediums, for storing computer-executable instructions, which, when executed by one or more computer processors 11, for example, can cause the computer processors 11 to perform the techniques described herein. The memory 8 optionally includes high-speed random-access memory, such as DRAM, SRAM, DDR RAM, or other random-access solid-state memory devices; and optionally includes non-volatile memory, such as one or more magnetic disk storage devices, optical disk storage devices, flash memory devices, or other non-volatile solid-state storage devices.

Further, the 3D bio-printer 100 may be connected to external network(s) 20, and remote servers 15 via for instance a wireless link or communication interface 14 via various technologies such as cellular long range or short range such as Wireless Local Area (LAN), Wi-Fi, etc. communication technologies.

The present disclosure has been presented above with reference to specific embodiments. However, other embodiments than the above described are possible and within the scope of the disclosure. Different method steps than those described above, performing the method by hardware or software, may be provided within the scope of the disclosure.

For instance, according to an exemplary embodiment a cloud computing system 20 or the remote server 15 can be configured to perform any of the methods presented herein. The cloud computing system may comprise distributed cloud computing resources that jointly perform the methods presented herein under control of one or more computer program products.

The processor(s) 11 (associated with the control system 10) may be or include any number of hardware components for conducting data or signal processing, training and executing neural network algorithm or for executing computer code stored in memory 8. The system 10 may have an associated memory 8, and the memory 8 may be one or more devices for storing data and/or computer code for completing or facilitating the various methods described in the present description. The memory may include volatile memory or non-volatile memory. The memory 8 may include database components, object code components, script components, or any other type of information structure for supporting the various activities of the present description. According to an exemplary embodiment, any distributed or local memory device may be utilized with the systems and methods of this description. According to an exemplary embodiment the memory 8 is communicably connected to the processor 11 (e.g., via a circuit or any other wired, wireless, or network connection) and includes computer code for executing one or more of processes described herein.

It should be appreciated that the 3D bio-printer 100 may further comprise a sensor interface 13 which may also provide the possibility to acquire sensor data directly or via dedicated sensor control circuitry.

The sensor devices such as the imaging sensor device e.g. camera sensors may communicate with the control system 10 using a local network setup, Ethernet, optical fibres, and so on.

As used herein, the term “if” may be construed to mean “when or “upon” or “in response to determining or “in response to detecting” depending on the context. Similarly, the phrase “if it is determined” or “in an instance of”, or “when it is determined” may be construed to mean “upon determining or “in response to determining” or “upon detecting and identifying occurrence of an event” or “in response to detecting occurrence of an event” depending on the context. The term “obtaining” is herein to be interpreted broadly and encompasses receiving, retrieving, collecting, acquiring, and so forth directly and/or indirectly between two entities configured to be in communication with each other or with other external entities.

Accordingly, it should be understood that parts of the described methods and systems may be implemented either in the 3D bio-printer, in a system located external the 3D bio-printer, or in a combination of internal and external the 3D bio-printer; for instance, in the server 15 in communication with the 3D bio-printer. For instance, sensor data may be sent to the external server 15 and that control system 15b of the server 15 performs the steps of the methods herein. The different features and steps of the embodiments may be combined in other combinations than those described.

It should be noted that the word “comprising” does not exclude the presence of other elements or steps than those listed and the words “a” or “an” preceding an element do not exclude the presence of a plurality of such elements. It should further be noted that any reference signs do not limit the scope of the claims, that the disclosure may be at least in part implemented by means of both hardware and software, and that several “means” or “units” may be represented by the same item of hardware.

Although the figures may show a specific order of method steps, the order of the steps may differ from what is depicted. In addition, two or more steps may be performed concurrently or with partial concurrence. In some embodiments and aspects several method steps may be carried out repetitively e.g. in feedback loops. Such variation will depend on the software and hardware systems chosen and on designer choice. All such variations are within the scope of the disclosure. Likewise, software implementations could be accomplished with standard programming techniques with rule-based logic and other logic to accomplish the various connection steps, processing steps, comparison steps and decision steps. The above mentioned and described embodiments are only given as examples and should not be limiting to the present disclosure. Other solutions, uses, objectives, and functions within the scope of the disclosure as claimed in the below described patent embodiments should be apparent for the person skilled in the art.

1. A computer-implemented method for calibrating a dispensing means of a 3D bio-printer, the method comprising:
obtaining sensor data representative of a printhead of the 3D bio-printer, and a surrounding environment of the printhead, the obtained sensor data comprising one or more images of the printhead of the 3D bio-printer captured by means of an imaging sensor device; wherein the sensor data comprises one or more sensor data points corresponding to the captured one or more images;
providing the obtained sensor data to a trained neural network algorithm, wherein the method further comprises:
determining, by means of the neural network algorithm, a presence of the dispensing means on the printhead based on the obtained sensor data; and
in an instance that the presence of the dispensing means is determined:
determining, by means of the neural network algorithm, an association of the dispensing means to one or more respective target object classes based on one or more characterizing features of the dispensing means;
determining, at least partly by means of the neural network algorithm, an operational state of the dispensing means associated with the respective one or more target object classes based on a comparison with a quality-of-state classification scheme; and
determining, by means of the neural network algorithm, corresponding spatial coordinates of a dispensing outlet of the dispensing means with respect to a print bed of the 3D bio-printer; and
generating, by means of the neural network algorithm, one or more prediction outputs representative of a calibration condition of the dispensing means based on the determined association of the dispensing means to one or more respective target object classes, the determined operational state of the dispensing means, and the determined spatial coordinates of the dispensing outlet of the dispensing means.

2. The method according to claim 1, wherein the dispensing means comprises any one of a dispensing needle type or a dispensing nozzle type.

3. The method according to claim 1, wherein the trained neural network algorithm comprises a convolutional neural network and/or a transformer neural network algorithm configured for determining one or more calibration conditions of the dispensing means.

4. The method according to claim 1, wherein each target object class is associated with one or more annotated data sets representative of that target object class, and wherein the one or more annotated data sets are representative of at least one annotated characterizing feature of the dispensing means;
wherein the one or more characterizing features comprise any one of a type of dispensing means, a colour indicator of the dispensing means, a size or a length measure of the dispensing means, or a transparency measure of the dispensing means.

5. The method according to claim 1, wherein the quality-of-state classification scheme comprises one or more quality-of-state classes, each class being associated with one or more annotated data sets representative of a respective quality of the dispensing means, and wherein the method further comprises:
determining the operational state of the dispensing means by determining an association of the obtained sensor data with at least one quality-of-state class.

6. The method according to claim 1, wherein the one or more obtained images comprise an ambient lighting and/or a shadow profile of the printhead and/or of the dispensing means when being present on the printhead and/or of the surrounding environment of the printhead.

7. The method according to claim 6, wherein the ambient lighting and/or the shadow profile of the surrounding environment of the printhead comprises an ambient lighting and/or a shadow profile of one or more reference markers arranged in the surrounding environment of the printhead.

8. The method according to claim 6, wherein the method further comprises:
determining the corresponding spatial coordinates of the dispensing outlet of the dispensing means based at least partly on the ambient lighting and/or the shadow profile of the printhead and/or of the dispensing means when being present on the printhead and/or of the surrounding environment of the printhead comprising the ambient lighting and/or a shadow profile of the one or more reference markers.

9. The method according to claim 1, wherein the method further comprises:
providing the obtained sensor data to an image analysis algorithm configured to determine an orientation of the dispensing means based on the obtained sensor data.

10. The method according to claim 1, wherein the method further comprises:
determining, by means of the neural network algorithm, respective spatial coordinates of each sensor data point comprised in the sensor data; and
determining the corresponding spatial coordinates of the dispensing outlet of the dispensing means based on the determined respective spatial coordinates for at least one sensor data point.

11. The method according to claim 1, wherein the method further comprises:
determining, by means of the neural network algorithm, when a printing fluid in connection with the printhead is extruded from the outlet of the dispensing means; and
when it is determined that the printing fluid is extruded from the outlet of the dispensing means:
terminating the extrusion of the printing fluid; and
generating a prediction output representative of the calibration condition indicative of an achieved priming of the outlet of the dispensing means.

12. A computer-readable storage medium comprising instructions which, when executed by one or more processors of a computing device of a 3D bio-printer, causes the computing device to carry out the method according to claim 1.

13. A computer program product comprising instructions which, when the program is executed by one or more processors of a computing device of a 3D bio-printer, causes the computing device to carry out the method according to claim 1.

14. A system for calibrating a dispensing means of a 3D bio-printer, the system comprising processing circuitry configured for:
obtaining sensor data representative of a printhead of the 3D bio-printer, and a surrounding environment of the printhead, the obtained sensor data comprising one or more images of the printhead of the 3D bio-printer captured by means of an imaging sensor device; wherein the sensor data comprises one or more sensor data points corresponding to the captured one or more images;
providing the obtained sensor data to a trained neural network algorithm, wherein the processing circuitry is further configured for:
determining, by means of the neural network algorithm, a presence of the dispensing means on the printhead based on the obtained sensor data; and
in an instance that the presence of the dispensing means is determined:
determining, by means of the neural network algorithm, an association of the dispensing means to one or more respective target object classes based on one or more characterizing features of the dispensing means;
determining, at least partly by means of the neural network algorithm, an operational state of the dispensing means associated with the respective one or more target object classes based on a comparison with a quality-of-state classification scheme; and
determining, by means of the neural network algorithm, corresponding spatial coordinates of a dispensing outlet of the dispensing means with respect to a print bed of the 3D bio-printer; and
generating, by means of the neural network algorithm, one or more prediction outputs representative of a calibration condition of the dispensing means based on the determined association of the dispensing means to one or more respective target object classes, the determined operational state of the dispensing means, and the determined spatial coordinates of the dispensing outlet of the dispensing means.

15. A 3D bio-printer comprising:
one or more printheads, each printhead comprising a dispensing means having an outlet for extruding a printing fluid; and
a system according to claim 14 for calibrating the dispensing means of a 3D bio-printer.
